(19) United States
(12) Patent Application Publication
(10) Pub. No.: US 2009/0133294 A1
(43) Pub. Date: May 28, 2009

(54) Apparatus and Method for Stimulating at least one Human Foot in response to the Human's Stance

(76) Inventor: Ronald Frank Harwin, Richmond, CA (US)

Correspondence Address: GREGORY SMITH & ASSOCIATES, 3900 NEWPARK MALL ROAD, 3RD FLOOR, NEWARK, CA, 94560, US

(21) Appl. No.: 11/944,654

(22) Filed: Nov. 26, 2007

(51) Int. Cl. A43B 7/14; A43B 7/22; A61F 5/14

(52) U.S. Cl. 36/145; 36/140; 36/155; 36/88; 36/91

(57) ABSTRACT

A device is disclosed comprising a contour configured to engage a foot and supported by a response assembly, the response assembly configured to mechanically respond to pressure applied to the contour. The contour may comprise at least one stimulating mound configured to engage at least one non-medial arch of the foot and/or a heel ridge for dividing the heel from the rest of the foot. A control circuit is disclosed that may communicate with an orthopedic device to create a pressure reading used to control a point of response and/or a stimulating mound. The device may include a second contour configured to engage a second foot supported by a second response assembly. Methods of using the device are disclosed. The device may be an orthopedic device and/or a sports training device. A shoe insert and shoe are disclosed, each comprising a contour including at least one stimulating mound.

### TECHNICAL FIELD

This invention relates to devices that include a contour for engaging a human foot with at least one stimulating mound to engage a non-medial arch of the human foot and/or provide response assembly including at least two points of response to pressure from the human foot engaging the contour. The device may be a sports training device, orthopedic device, shoe insert or shoe.

### BACKGROUND OF THE INVENTION

Many people have suffered with chronic injuries and/or diseases that have affected their body for a long time. Their body tends to adapt and compensate. Many people may suffer from pain syndromes resulting from such compensations to injuries. The injuries and/or diseases can weaken the mechanical process of the body and/or disrupt the pattern of bodily movement, either of which often can lead to deformations of bone, nerves, joints and/or tissues connecting and/or moving the bones and/or joints.

### SUMMARY OF THE INVENTION

No one today could reasonably say that these prior art problems can be solved with a machine, and neither can the inventor. But after years of research he is disclosing in this application tools he has found to fundamentally aid the body's own processes to improve health and maintain function.

One embodiment of the invention includes a device comprising a contour configured to engage a foot and supported by a response assembly, the response assembly comprising at least two points of response configured to mechanically respond to pressure applied to the contour. The response assembly may further comprise a first point of response configured to be in front of at least one of the toes of the foot and a second point of response configured to be behind a transverse arch of the foot, where the transverse arch goes across the ball of the foot from the inside to outside of the foot.

As used herein, the inside of the foot is near the big toe and the outside of the foot is near the little toe. While this is common usage of the terms, this usage should not be construed to limit the scope of the invention to human feet having both big and little toes. Certain embodiments of the device may serve orthopedic purpose and be referred to as an orthopedic device, whereas other embodiments may serve a sports training purpose and be referred to as a sports training device. As used herein an orthopedic purpose will include treating and/or alleviating the cause and/or effect of deformities, diseases and/or injuries of bones, nerves, joints and/or the tissues connecting and/or moving bones and/or joints.

The contour may comprise at least one stimulating mound configured to engage at least one non-medial arch. The stimulating mound may be any of the following: a first stimulating mound to engage the transverse arch of the human foot, a second stimulating mound to engage a lateral arch of the human foot without arch support for a medial arch of the foot, a third stimulating mound to further engage the lateral arch in front of the second stimulating mound as well as outside and behind the first stimulating mound and a fourth stimulating mound to engage the transverse arch near the lateral arch.

As used herein, the lateral arch goes along the outside ridge of the foot extending from the ball to a heel of the foot. In many situations the stimulating mound may preferably including a yielding material that resiliently returns to a rest state when not engaged by the foot. In other situations the stimulating mound includes a more rigid material that does not noticeably deform when engaging the foot.

One embodiment of the invention includes a control circuit configured to communicate with a pressure sensor for the point of response to create a pressure reading, for at least one of the points of response and configured to control at least one of the stimulating mounds based upon at least one of the pressure readings. Preferably, the stimulating mound is controlled using a pneumatic device and/or a hydraulic device. The control circuit may be driven by electrical power, which may further power the pneumatic and/or hydraulic device. Alternatively, a pressure sensor for at least one stimulating mound may communicate a pressure reading to the control circuit and the control circuit may control at least one of the points of response through a pneumatic device or a hydraulic device. Any combination of pressure sensors for points of response and/or stimulating mounds may be communicating with the control circuit. Any combination of pneumatic devices and/or hydraulic devices for stimulating mounds and/or points of response may be controlled by the control circuit. In certain embodiments, at least one of the stimulating mounds and/or points of response may include one or more materials that respond to electrical, optical and/or thermal stimulus to change their mechanical properties. These changes may include changes in rigidity, softness, shape and/or mechanical resistance. The changes may be approximated by a force or a flow that may include a twist and/or thrust.

The contour may comprise a heel ridge configured to urge a separation of the heel from the rest of the foot. In certain embodiments, this may used to create greater mobility in the heel. A heel adjustment mechanism may be used to adjust the heel ridge position on the heel. Alternatively, a heel contour with the heel ridge may be used to adjust the heel ridge to the heel.

The device may further comprise a second contour for engaging a second foot supported by a second response assembly responding to the pressure on the second of the contours.

The human stands on the device to operate it. Imbalances in the pressure of the foot engage the contour and lead to responses both from the points of response, but also from the stimulating mounds. These responses in turn cause the human to shift the pressure of their feet on the contours, leading to different responses and so on. The inventor believes that the heel pressing the heel ridge causes the heel movement to urge separation between the heel and the foot, possibly allowing the heel to move downward, freeing the heel to move more freely, and counteracting the tendency of rigidity.

Our species tends to walk upright. It is surmised that the effect of using the device may include feedback stimulating the central nervous system through its monitoring of the foot-ankle relationship with what is perceived of as ground. The process of walking upright appears to be stimulated through this, affecting the balance mechanism through the foot-ankle relationship.

The device is used by a human performing the following steps: The human stands on it by placing at least one of their feet on a contour and the response assembly providing feedback to the pressure on the contour for a first time interval. The human then performs an activity off the device for a second time interval. The human may further stand on the device for a third time interval. Alternatively, an activity may first be performed off of the device and then the device may be stood upon. These activities may be part or all of a prescribed sequence of exercises with possibly an athletic and/or therapeutic objective. The device may be used with the contour essentially horizontal, the foot may have the toes below the heel, or the heel below the toes.

The device may further be used by a succession of humans before performing the same activity, such as an athletic training and/or rehabilitation session. Or people may subsequently perform independent activities. The device may be used in a nursing home and/or an outpatient clinic and/or a home.

Another embodiment of the invention includes a shoe insert, comprising at least one contour for engaging a foot of a human, comprising at least one stimulating mound configured to engage at least one non-medial arch of the foot similar to the above description of the contour for the orthopedic device.

Another embodiment of the invention includes a shoe whose insoles provide the contours as described above. The shoe pair may be a boot pair, a lace-up shoe pair, a sandal pair, and a mule pair.

### BRIEF DESCRIPTION OF THE DRAWINGS

FIGS. 1A to 1E show the top or side cutaway views of examples of a device comprising a contour for engaging a foot supported by a response assembly, the response assembly comprising two points of response configured to mechanically respond to pressure applied to the contour, where the points of response are of various sizes and at various locations in accord with the invention.

FIGS. 1F and 1G show the top view and side cutaway view of the device, where the response assembly includes more than two points of response.

FIG. 2A shows a top view of an example of a preferred contour comprising at least one stimulating mound configured to engage at least one non-medial arch as used in embodiments of the device and/or the shoe insert.

FIG. 2B shows preferred contour may include any of the following: a first stimulating mound to engage the transverse arch, a second stimulating mound to engage a lateral arch without arch support for a medial arch, a third stimulating mound to further engage the lateral arch in front of the second stimulating mound behind and outside of the first stimulating mound, and a fourth stimulating mound to engage the transverse arch near the lateral arch.

FIGS. 3A to 3F show examples of a mound adjustment mechanism for positioning at least one of the stimulating mounds.

FIGS. 4A and 4B show simplified schematics of examples of a control circuit communicating with pressure sensors to create a pressure reading used to control at least one of the stimulating mounds and/or at least one of the points of response based upon at least one of the pressure readings.

FIG. 5A shows the contour may further comprise a heel ridge configured to urge the heel to separate the rest of the foot.

FIG. 5B shows the contour of FIG. 5A with the heel ridge configured to urge the front and back of the heel to separate.

FIG. 5C shows an example of the heel ridge including at least two heel ridge components configured to collectively urge the heel to separate from the rest of the foot.

FIG. 5D shows the heel ridge 60 configured to adjust the heel ridge, preferably through the use of a heel adjustment mechanism.

FIG. 5E shows a heel contour including the heel ridge configured to adjust the position of the heel ridge.

FIG. 6A shows an example of the device further comprising a second contour configured to engage a second foot supported by a second response assembly responding to the pressure on the second contour, with both the first and the second response assembly rigidly coupled through a base plate.

FIG. 6B shows an example of at least one of the response assemblies configured to stack on top of the other response assembly.

FIG. 7 shows an example of a shoe whose insole provides the contour as described in previous Figures.

### DETAILED DESCRIPTION

This invention relates to devices that include a contour for engaging a human foot with at least one stimulating mound to engage a non-medial arch of the human foot and/or provide response assembly including at least two points of response to pressure from the human foot engaging the contour. The device may be a sports training device, orthopedic device, shoe insert or shoe.

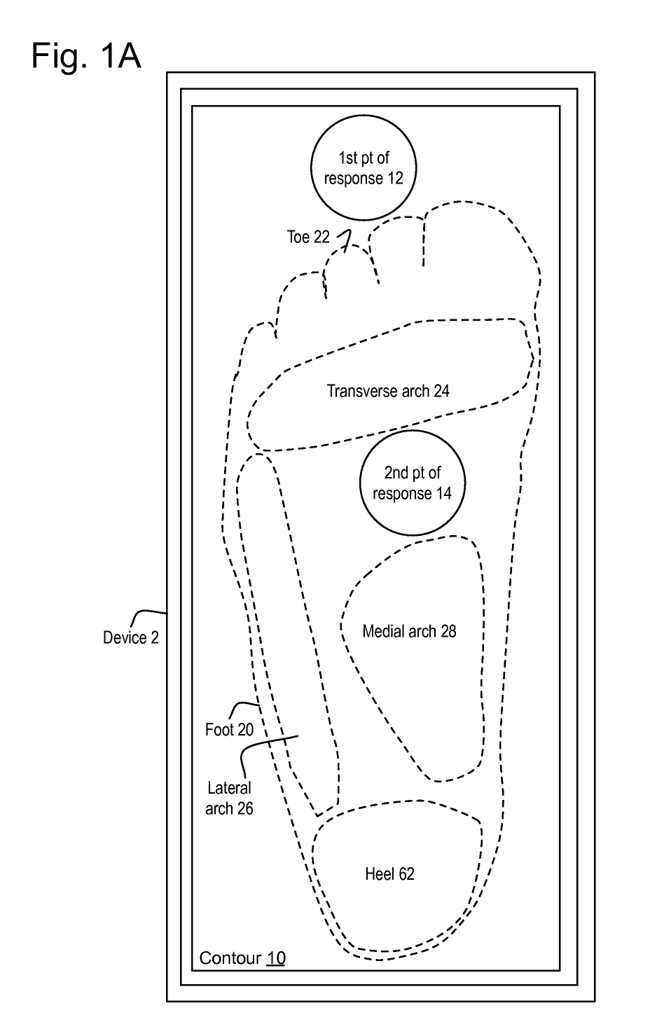

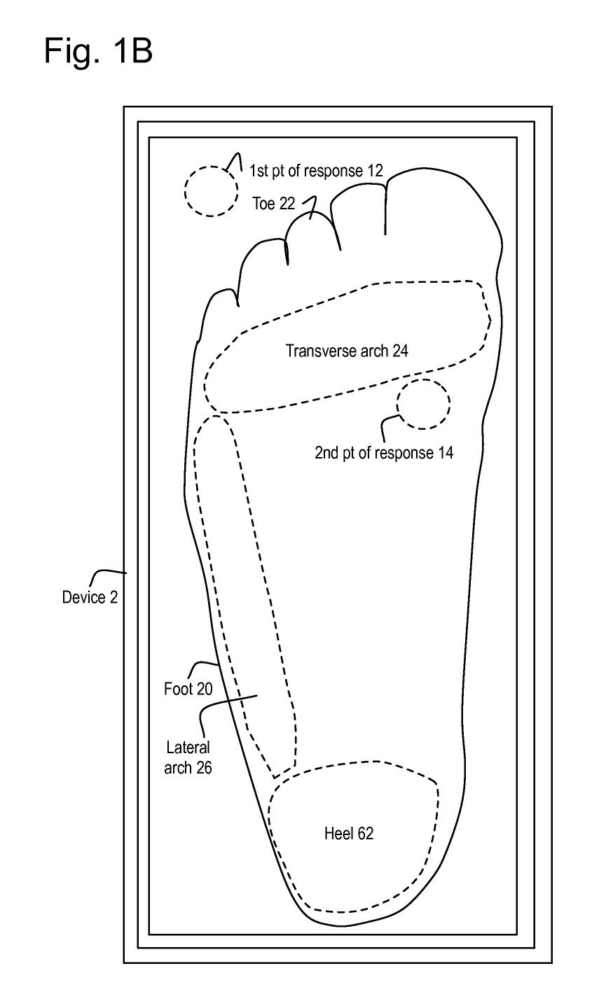

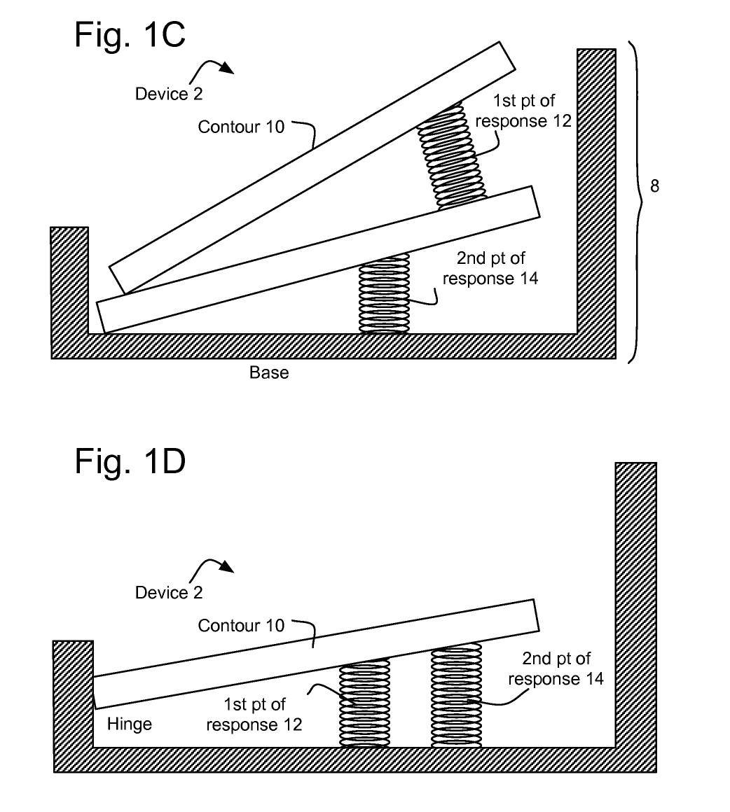

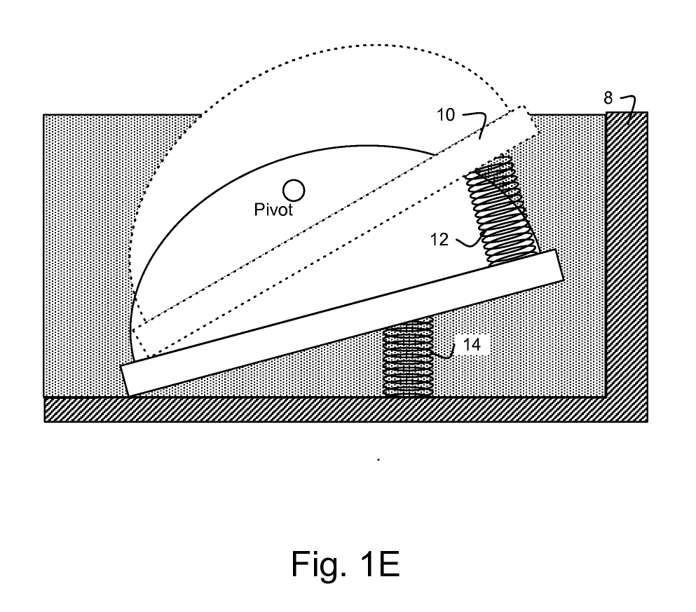

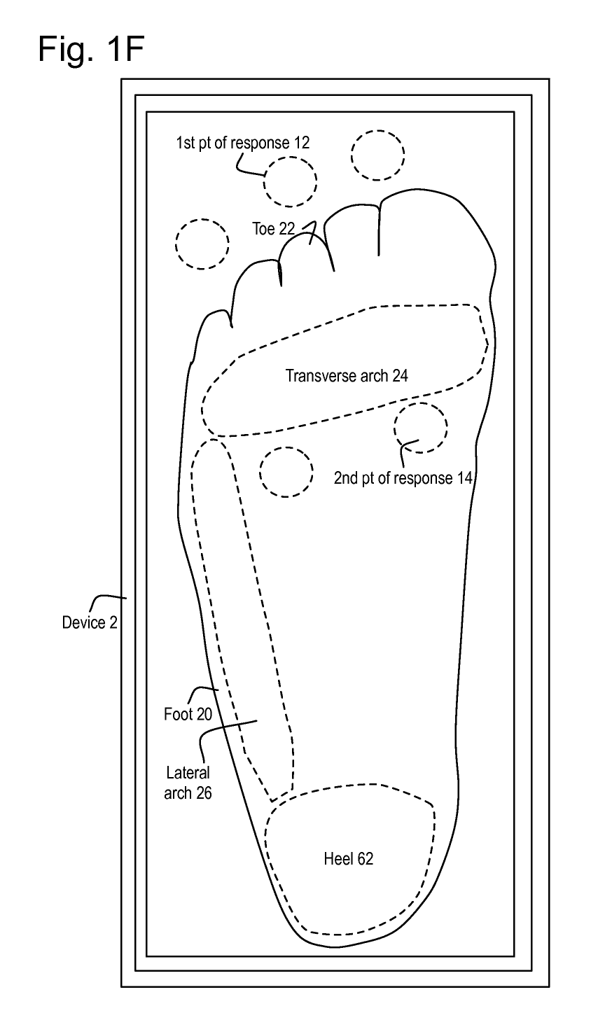

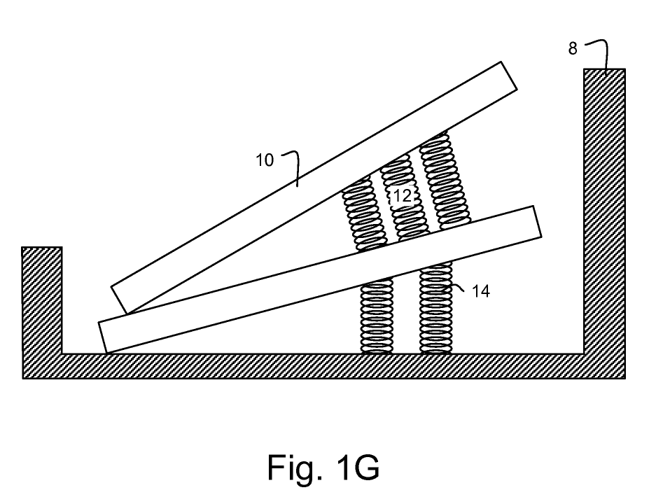

FIGS. 1A to 1E show the top or side cutaway views of example embodiments of the invention as a device 2 comprising a contour 10 for engaging a foot 20 supported by a preferred response assembly 8, the response assembly comprising at least two points of response 12 and 14 configured to mechanically respond to pressure applied to the contour. These examples show the points of response in varying locations and varying sizes. The first point of response 12 is configured to be in front of at least one toe 22 of the foot and a second point of response 14 configured to be on or behind a transverse arch 24 of the foot, where the transverse arch goes across a ball of the foot from the inside to the outside of the foot.

FIGS. 1C to 1E show some alternative embodiments of the response assembly 8 of FIGS. 1A and 1B.

FIG. 1C shows the contour 10 contacting the first point of response 12 mounted on a second plane, which also contacts the contour. The second plane contacts the second point of response 14 and a base of the response assembly. The points of response are shown as springs and/or elastic devices.

FIG. 1D shows the contour 10 contacting both points of response 12 and 14 mounted on a base of the response assembly 8. The contour 10 preferably pivots about the response assembly, preferably through a hinge or bearing shown on the left wall.

FIG. 1E show the contour 10 mounted on a first disk pivotably connected to a second disk. The first point of response 12 is connected to the contour and to a second plane coupled to the second disk. The second plane is connected to the second point of response 14 and the base of the response assembly 8. This is an example of a response network that may move the contour as whole at least some of the time. Other embodiments may include the ability to position the contour with respect to the response assembly.

FIGS. 1F and 1G show the top and side cutaway views of an example of the device 2 with more than two points of response. As before, the first point of response 12 is configured to be in front of at least one toe 22 of the foot and a second point of response 14 configured to be on or behind a transverse arch 24 of the foot. There are two other points of response, each in front of at least one of the toes and one additional point of response behind the transverse arch. In certain embodiments, the front points of response may be on top of the points of response on or behind the transverse as shown in FIG. 1G, but in other embodiments, this relationship may be reversed.

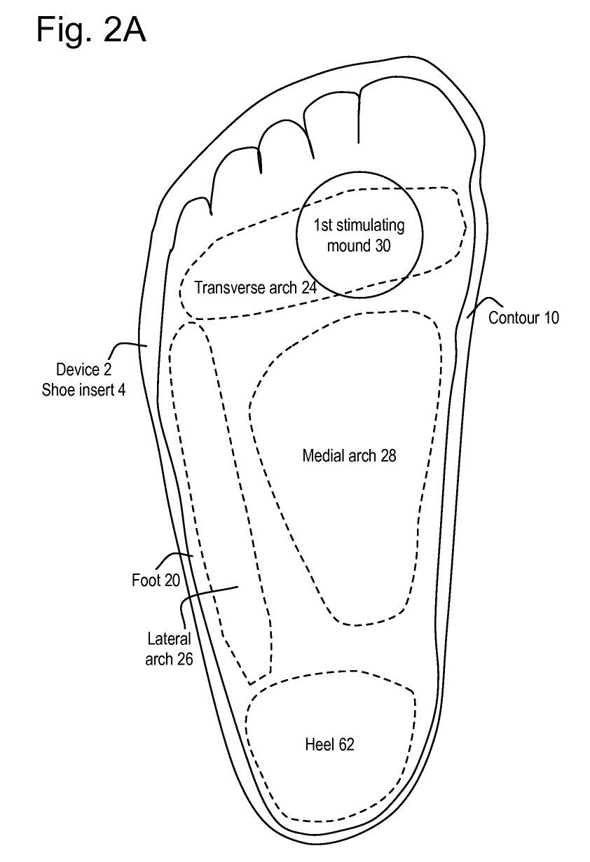

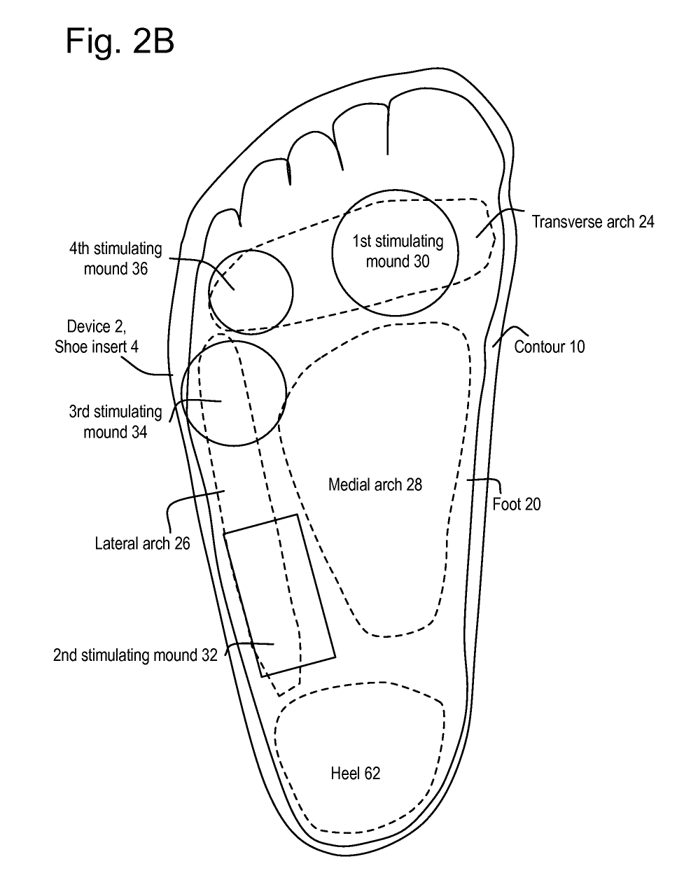

FIG. 2A shows a top view of an example of a preferred contour 10 comprising at least one stimulating mound 30 configured to engage at least one non-medial arch as used in the orthopedic device 2 and/or as used in a shoe insert 4. As used herein, a non-medial arch includes the transverse arch 24, the lateral arch 26 and the arch found in a toe 22. The medial arch 28 is not included in any of the non-medial arches of the human foot. In many embodiments of the invention, the stimulating mounds may include a yielding material that is resilient.

FIG. 2B shows the contour 10 may include any of the following: a first stimulating mound 30 to engage the transverse arch 24 of the human foot 20, a second stimulating mound 32 to engage a lateral arch 26 of the human foot, preferably without arch support for a medial arch 28 of the foot, the lateral arch going along an outside ridge of the foot extending from the ball 16 to a heel 62 of the foot, a third stimulating mound 34 to further engage the lateral arch in front of the second stimulating mound, behind and outside of the first stimulating mound, and a fourth stimulating mound 36 to stimulate the lateral side of the transverse arch. The contour may include two or more of these stimulating mounds.

A stimulating mound may preferably include a yielding material that resiliently returns to a rest state when not engaged by the foot. Alternatively the stimulating mound may include a more rigid material that does not noticeably deform when engaging the foot and may in such situations be shallow compared to a stimulating mound including the yielding material.

The size and shape of the stimulating mounds may vary not only as shown in these Figures, but also in other ways not illustrated in these Figures. By way of example, the stimulating mounds may not only be oval, circular or polygonal, one or more of them may also be convex or concave. A shape will be referred to as convex is any two points within it may be connected by a straight line segment contained within the shape. A shape will be referred to as concave if there are two points within that shape which cannot be connected by a straight line segment contained within the shape.

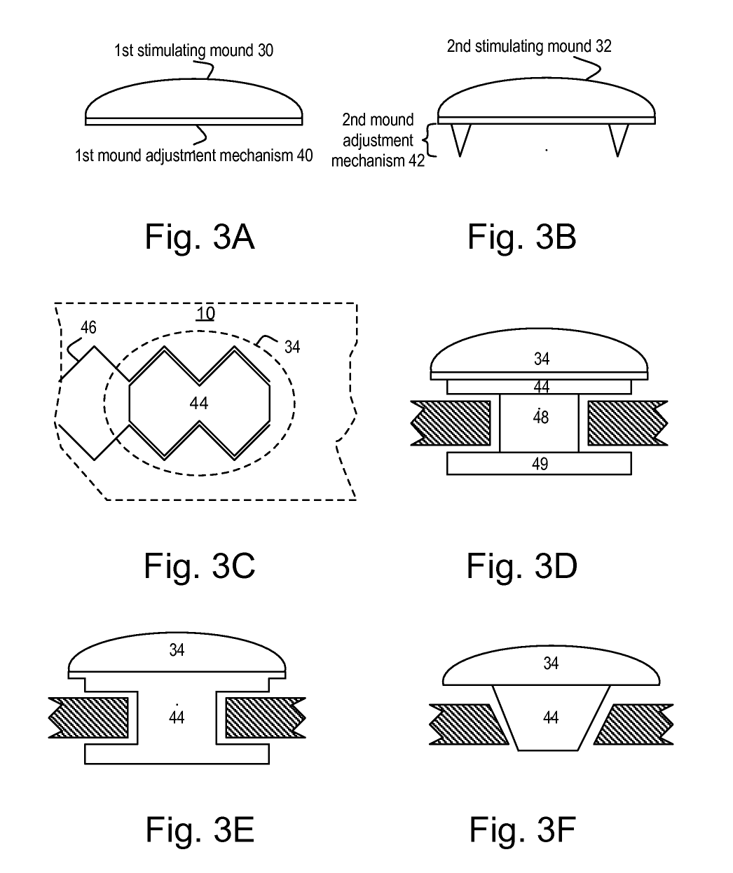

FIGS. 3A to 3F show some examples of a mound adjustment mechanism for positioning at least one of the stimulating mounds.

FIG. 3A shows the device and/or the shoe insert may further include a mound adjustment mechanism 40 for positioning at least one of the stimulating mounds, for example, the first stimulating mound 30. In this example, the mound adjustment mechanism may include an adhesive surface used to attach it to the contour 10.

FIG. 3B shows a second mound adjustment mechanism 42 for positioning the second stimulating mound 32 and including at least one and preferably two prongs for insertion into the contour 10, where the contour is composed of a material such as cork supporting both easy insertion and sufficient strength. Other examples may include a contour with a collection of holes or tracks into which the second mound adjustment mechanism may be inserted to position the second stimulating mound.

FIG. 3C shows a third mound adjustment mechanism 44 for positioning the third stimulating mound 34 and further including a narrow throat 48 coupled to the third adjustment mechanism and may also be coupled to a wide base 49. The third stimulating mound is positioned in a contour mound adjustment track 46 by pressing the mound down, causing the adjustment mechanism to lock into the adjustment track. To reposition the mound, it is lifted until the narrow throat is between the sides of the mound adjustment track as shown in FIG. 3D, which allows the mound to be moved along the track. FIGS. 3E and 3F show alternative to the mound adjustment mechanism as a single piece coupled to the third stimulating mound.

At least one of the stimulating mounds 30, 32, and/or 34 may include an elastic device. By way of example, in certain prototypes, parts of rubber balls were used as the stimulating mounds. In other prototypes, the stimulating mounds were formed from various forms of plastic such as foam.

Each point of response 12 and/or 14 may be driven by a spring 18, which may be a compressive spring or an expansion spring. Alternatively the point of response may be driven by a hydraulic device 58, or a pneumatic device 56. The response assembly 8 may further include a hinge 36 and/or a pivot coupling essentially to the floor located near one of the following: the heel 62, the toes 22, the lateral arch 26, or the medial arch 28.

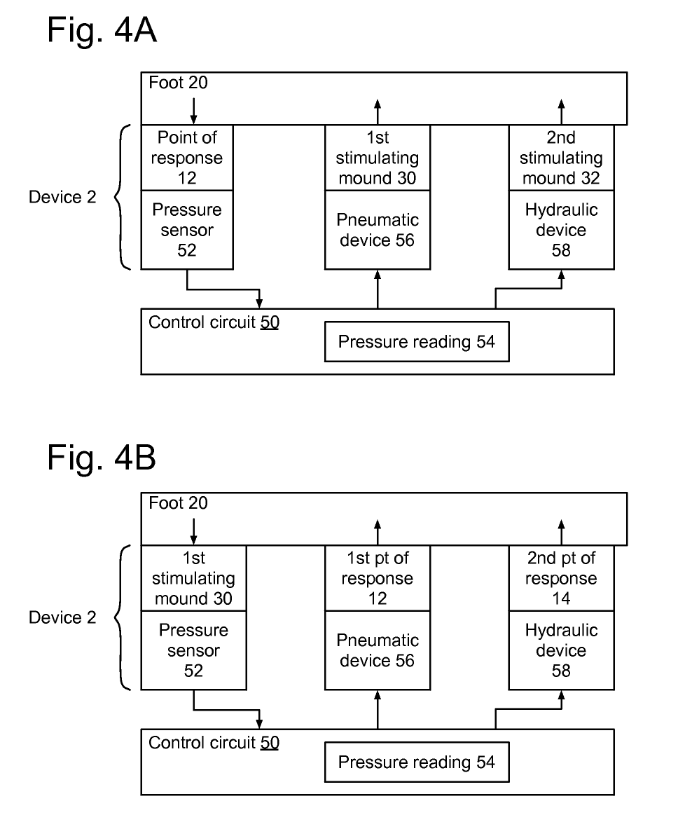

FIGS. 4A and 4B show simplified schematics of examples of a control circuit 50 communicating with at least one pressure sensor 52 to create a pressure reading 54 used to controllably direct the alteration of at least one of the stimulating mounds 30, 32, 34 and/or 36 and/or at least one of the points of response 12 and 14 based upon at least one of the pressure readings. The at least one pressure sensor may or may not be coupled to a stimulating mound or a point of response. The following examples are not meant to limit the scope of the claims, but instead to provide simple examples of embodiments of the invention.

FIG. 4A shows the control circuit 50 communicating with the pressure sensor 52 for the point of response 12 to receive the pressure reading 54 used in controlling the first stimulating mound through a pneumatic device 56 and/or in controlling the second stimulating mound 32 through a hydraulic device 58 based upon at least one of the pressure readings.

FIG. 4B shows the control circuit 50 communicating with the pressure sensor 52 for at least one stimulating mound 30 to create the pressure reading 54 used to control the first point of response 12 through the pneumatic device 56 and/or to control the second point of response 14 through the hydraulic device 58 based upon at least one of the pressure readings.

Any combination of pressure sensors 52 for points of response 12, 14 and/or stimulating mounds 30, 32, 34 and/or 36 may be communicating with the control circuit 50. The control circuit may controllably direct any combination of pneumatic devices 56 and/or hydraulic devices 58 for stimulating mounds and/or points of response. Typically, it will be preferred to power the control circuit with electrical power, which may be provided by a battery pack and/or an external power source. In certain embodiments, the altering of a stimulating mound or point of response may use a material whose properties are altered by electrical, optical and/or thermal stimulus as directed by the control circuit.

The device 2 may include pressure sensors 52 and/or pneumatic devices 26 and/or hydraulic devices 58 as shown in the examples of FIGS. 4A and 4B.

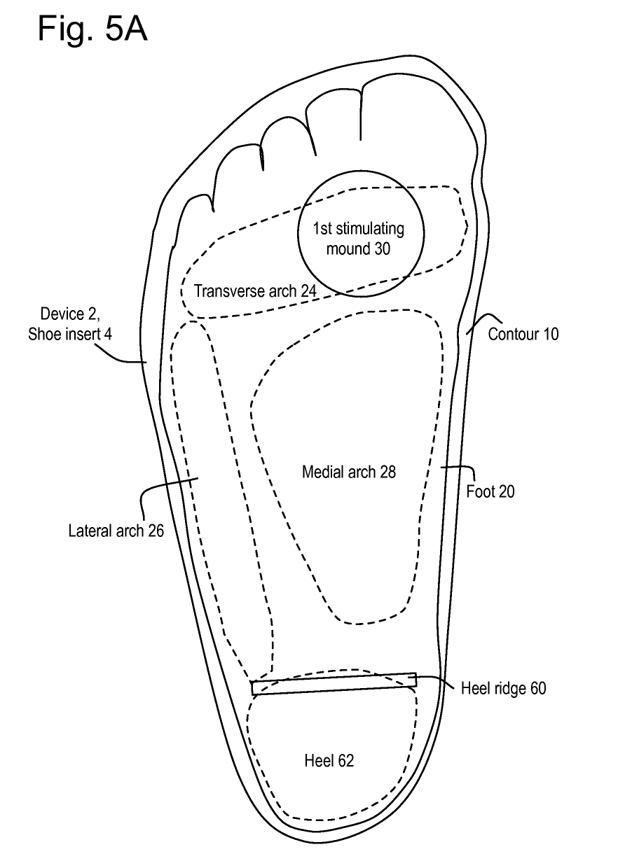

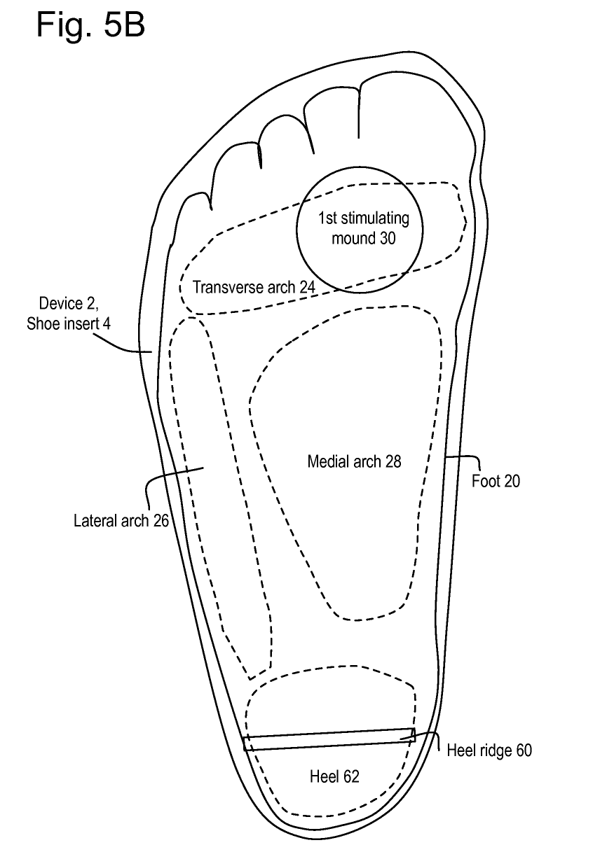

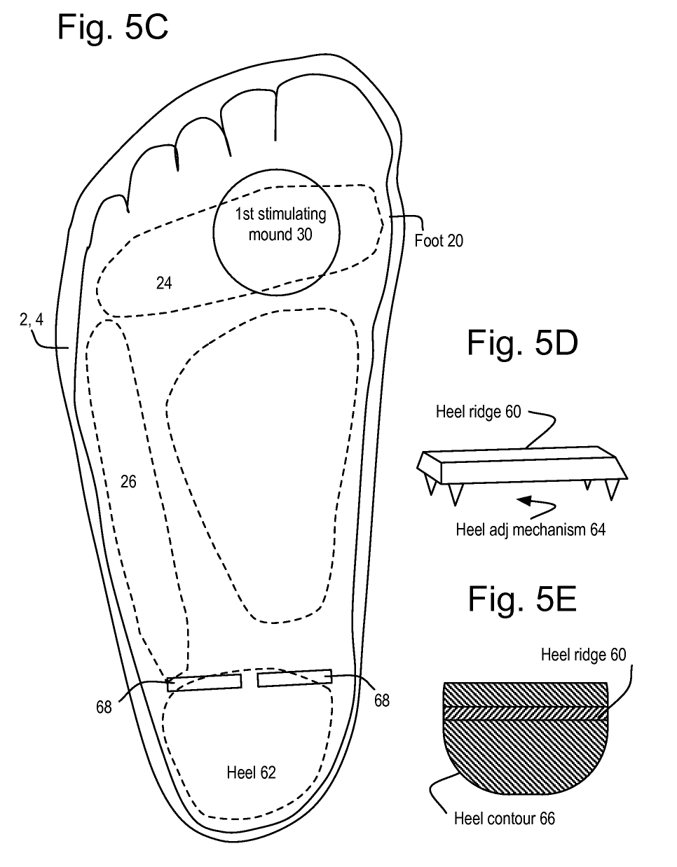

FIG. 5A an example of the device 2 and/or a shoe insert 4 where the contour 10 may further comprise a heel ridge 66 configured to urge the heel 62 to separate from the rest of the foot 20.

FIG. 5B shows an example of the device 2 and/or the shoe insert 4 where the heel ridge urges the separation of the heel 62 from the rest of the foot 20.

FIG. 5C shows an example of the device 2 and/or the shoe insert 4 where the heel ridge 60 including at least two heel ridge components 68 configured to collectively urge the heel 62 to separate from the foot 20. These components are shown in a collinear arrangement, but may be arranged in any other configuration that collectively urges the heel to separate from the foot.

FIG. 5D shows an example of a heel adjustment mechanism 64 to adjust the heel ridge 60 through the insertion or removal of cleats into the contour 10.

FIG. 5E shows a heel contour 66 including the heel ridge 60 configured to adjust the heel ridge, preferably through the use of an adhesive backing.

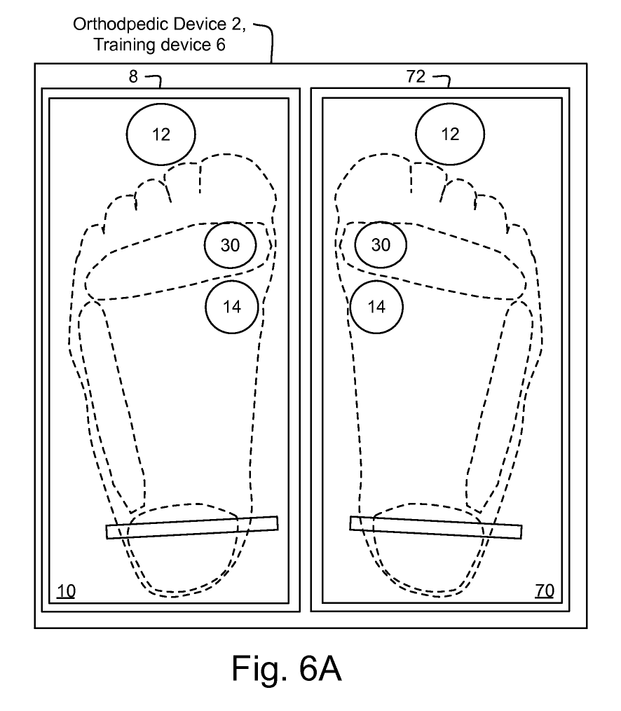

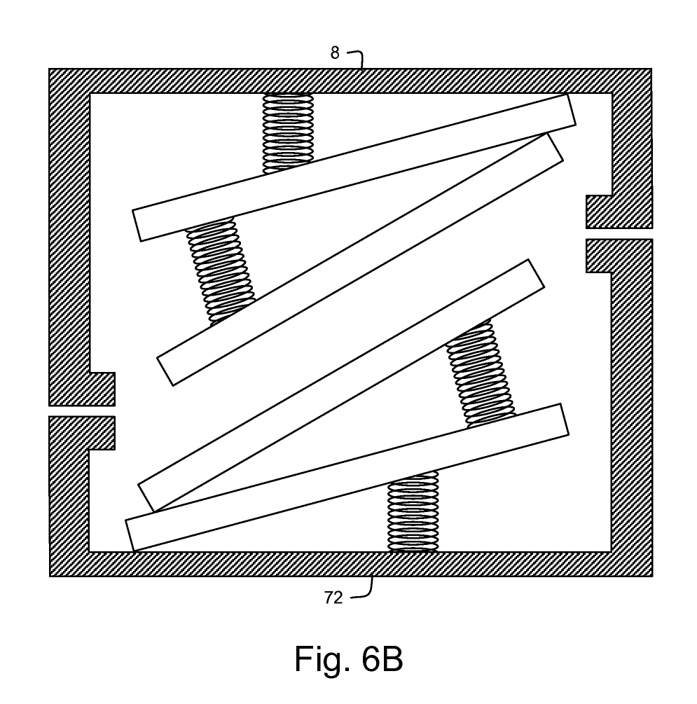

FIG. 6A shows the orthopedic device 2 and/or the training device 6 may further comprise a second contour 70 configured to engage a second foot 20 supported by a second response assembly 72 responding to the pressure on the second contour, both the first response assembly 8 and the second response assembly 72 are rigidly coupled through a base plate 74.

FIG. 6B shows an example at least one of the response assemblies 74 configured to stack on top of the other response assembly 8.

The human stands on the device 2 to operate it. Imbalances in the pressure of the foot 20 engage the contour 10 and lead to responses both from the points of response 12 and 14, but also from the stimulating mounds 30, 32, 34 and/or 36. These responses in turn cause the human to shift the pressure of their feet on the contours, leading to different responses and so on. The shifting of the pressure of their feet is not necessarily conscious. The heel 62 pressing into the heel ridge 60 urges the heel movement to separate the heel from the rest of the foot, with the back of the heel moving down. It is believed that this frees the heel to move more freely, counteracting the heel's commonly observed tendency of rigidity.

Our species tends to walk upright. It is surmised that the effect of using the device 2 may include feedback stimulating the central nervous system through its monitoring of the foot-ankle relationship with what is perceived of as ground. The process of walking upright appears to be stimulated through this, affecting the balance mechanism through the foot-ankle relationship.

The device 2 may be used with the contour 10 essentially horizontal. Alternatively, by contour may be configured so the foot 20 may have the toes 22 below the heel 62, or the heel below the toes.

The device 2 may be preferably used by a human performing the following steps: The human stands on it by placing at least one of their feet 20 on a contour 10 and the response assembly 8 provides feedback to the pressure on the contour for a first time interval. The human then performs an activity off of the device for a second time interval. The human may further stand on the device for a third time interval. Alternatively, an activity may first be performed off of the device and then the device may be stood upon.

The first time interval may be at least five seconds and may further be less than three minutes. The first time interval may further, preferably, be at least fifteen seconds. The second time interval may be at least ten minutes and may further be less than four hours. The third time interval may be at least five seconds and may further be less than three minutes. The third time interval may also preferably be at least fifteen seconds.

The activity off of the device 2 may be a form of walking. Alternatively, the activity may be a form of athletic exercise and/or a rehabilitative exercise. The athletic exercise may be any of a form of dance, basketball, baseball, tennis, football, rugby, soccer, track and field event training, swimming, horseback riding, rowing, martial art, cricket and so on. The rehabilitative exercise may be designed to restore movement or strength after an injury.

The orthopedic device 2 may further be used by a succession of humans before performing the same activity, such as an athletic training session and then used by those humans after the activity. Alternatively, the device may be used by a succession of people who then perform their activities independently, such as in a nursing home and/or an outpatient clinic and/or a private home.

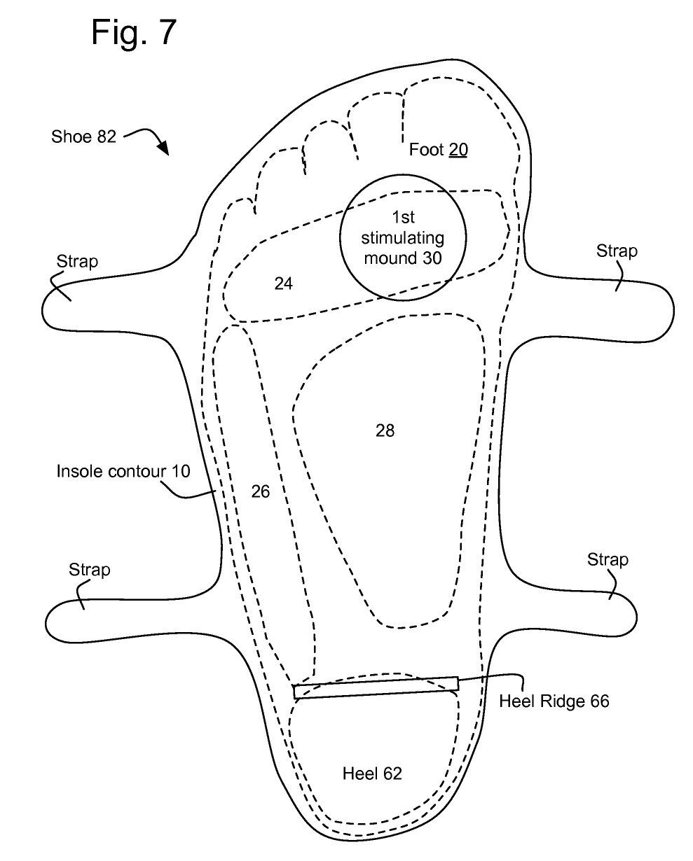

FIG. 7 shows an example of a shoe 82 whose insole provides the contour 10 as described in previous Figures. The shoe may be a boot, a lace-up shoe using laces and/or hook and eye straps to secure the shoe to the foot, a sandal as shown with straps as shown which are coupled to secure the shoe on the foot 20, and a mule providing a toe binding to at least partly secure the shoe. The straps may be in any configuration desired to secure the foot while being worn.

Various embodiments of the invention may also include a support for the medial arch 28.

Aspects of this invention, in particular the points of response may be used to implement similar embodiments that may be beneficial for animals, whether two legged or four legged, offering a potential for veterinary application.

The preceding embodiments provide examples of the invention and are not meant to constrain the scope of the following claims.

What is claimed is:

1. An device, comprising: at least one contour configured to engage at least one foot of a human supported by a response assembly, said response assembly comprising at least two points of response configured to mechanically respond to pressure applied to said contour.

2. The device of claim 1, wherein said response assembly further comprises a first of said points of response configured to be in front of at least one of the toes of said foot and a second of said points of response configured to be on or behind a transverse arch of said foot.

3. The device of claim 1, wherein said contour comprises at least one stimulating mound configured to engage at least one non-medial arch.

4. The device of claim 3, wherein said stimulating mound is member of the stimulating mound group consisting of:
a first stimulating mound configured to engage said transverse arch of said human foot; and
a second stimulating mound configured to engage a lateral arch of said human foot without arch support for a medial arch of said foot, said lateral arch going along an outside ridge of said foot extending from said ball to a heel of said foot;
a third stimulating mound configured to further engage said lateral arch in front said second stimulating mound behind and outside of said first stimulating mound; and
a fourth stimulating mound configured to further engage said transverse arch near said lateral arch.

5. The device of claim 3, wherein said contour further comprises a mound adjustment mechanism to position at least one of said stimulating mounds.

6. The device of claim 3, wherein at least one of said stimulating mounds includes an elastic device.

7. The device of claim 1, wherein said contour, comprises:
a heel ridge configured to urge said heel to separate from said foot.

8. The device of claim 7, said heel ridge further comprises at least two heel ridge components.

9. The device of claim 7 further comprises at least one of
a heel adjustment mechanism to adjust said heel ridge to said heel of said foot and
a heel contour including said heel ridge configured to adjust said heel ridge to said heel of said foot.

10. The device of claim 1, wherein said device is at least one of an orthopedic device and a sports training device.

11. The device of claim 1 further comprising a second of said contours configured to engage a second foot of said human supported by a second of said response assemblies responding to said pressure on said second of said contours.

12. The device of claim 11, further comprising a base plate rigidly coupling said response assembly and said second of said response assemblies.

13. The device of claim 11, wherein at least one of said response assemblies is configured to stack on top of the other of said response assemblies.

14. A control circuit, comprising:
said control circuit configured to interact with a device, comprising:
said control circuit configured to communicate with at least one pressure sensor to create a pressure reading based upon pressure applied by a foot engaging a contour; and
said control circuit configured to controllably direct to at least one of
at least one stimulating mound to alter said stimulating mound based upon at least one of said pressure readings; and
at least one point of response to alter said point of response based upon at least one of said pressure readings.

15. The device for interacting with said control circuit of claim 14, comprising:
a contour configured to engage a foot supported by a response assembly, for at least one foot of a human, said contour comprising at least one of said stimulating mounds configured to engage at least one non-medial arch of said foot; and
said response assembly comprises at least one of said points of response configured to mechanically respond to pressure applied to said contour.

16. The control circuit of claim 14, comprises at least one instance of a controller, each of said instances receives at least one input, maintains and updates at least one state and generates at least one output based upon the value of at least one of said inputs and said states.

17. A device, comprising:
a contour for engaging a foot supported by a response assembly, for each of said foot of a human,
each of said response assemblies comprises at least two points of response configured to mechanically respond to pressure applied to said contour, and
each of said contours for engaging said foot comprises at least one stimulating mound configured to engage at least one non-medial arch of said foot, and a heel ridge configured to urge the heel to separate from said foot.

18. The device of claim 17, wherein each of said contours comprises
a first stimulating mound to engage said transverse arch of said foot; and
a second stimulating mound to engage a lateral arch of said foot, said lateral arch going along an outside ridge of said foot extending from said ball to a heel of said foot;
a third stimulating mound to engage said lateral arch in front said second stimulating mound behind and outside of said first stimulating mound; and
a fourth stimulating mound to engage said transverse arch near said lateral arch.

19. The device of claim 17, wherein each of said contours further comprises a mound adjustment mechanism configured to position at least one of said stimulating mounds.

20. The device of claim 17, further comprising a base plate rigid coupling said first and said second of said response assemblies.

21. The device of claim 17, wherein at least one of said response assemblies is configured to stack on top of the other of said response assemblies.

22. A method, comprising the steps of:
at least one human using a device by placing a foot to stand on a contour supported by a response assembly providing mechanical feedback to the pressure of said foot on said contour for a first time interval; and
said human performs an activity off of said device for a second time interval after said first time interval.

23. The method of claim 22, wherein said first time interval is at least five seconds and said second time interval is at least ten minutes.

24. The method of claim 22, wherein said activity is at least one of a form of walking, an athletic exercise, and a rehabilitative exercise.

25. The method of claim 22, wherein the step of said human using said device further comprises the step of
the human placing a second foot on a second contour supported by a second response assembly providing mechanical feedback to the pressure of said second foot on said second contour for essentially said first time interval.

26. The method of claim 22, further comprising the step of:
said human using said device by placing said foot to stand on said contour for a third time interval after said second time interval.

27. The method of claim 22, further comprising the steps of:
a succession of at least two of said humans using said device; and said method further comprises at least one of the steps of:
said humans performing a same activity after using said device; and
said humans performing said activity independently after using said device.

28. The method of claim 27, wherein said same activity is at least one of an athletic training session and a rehabilitative training session.

29. A method, comprising the steps of:
at least one human performs an activity off of a device for a second time interval; and
said human using said device by placing a foot to stand on a contour supported by a response assembly providing mechanical feedback to the pressure of said foot on said contour for a third time interval after said second time interval.

30. The method of claim 29, further comprising the step of:
said human using said device by placing said foot to stand on said contour for a first time interval before said second interval.

31. A shoe insert, comprising: at least one contour configured to engage a foot of a human, comprising at least one stimulating mound configured to engage at least one non-medial arch of said foot.

32. The shoe insert of claim 31, wherein said stimulating mound is a member of the stimulating mound group consisting of:
a first stimulating mound to engage said transverse arch of said human foot; and
a second stimulating mound to engage a lateral arch of said human foot, said lateral arch going along an outside ridge of said foot extending from said ball to a heel of said foot;
a third stimulating mound to further engage said lateral arch in front said second stimulating mound behind and outside of said first stimulating mound; and
a fourth stimulating mound to engage said transverse arch near said lateral arch.

33. The shoe insert of claim 31, further comprising: a second of said contours for engaging a second foot of said human.

34. The shoe insert of claim 31, wherein at least one of said stimulating mounds includes an elastic device.

35. The shoe insert of claim 31, wherein said contour, further comprises: a heel ridge configured to urge said heel to separate from said foot.

36. The shoe insert of claim 35, said heel ridge further comprises at least two heel ridge components configured to urge said heel to separate from said foot.

37. A shoe, comprising: at least one contour comprising at least one stimulating mound configured to engage at least one non-medial arch of a foot of a human.

38. The shoe of claim 37, wherein said stimulating mound is member of the stimulating mound group consisting of:
a first stimulating mound to engage said transverse arch of said foot; and
a second stimulating mound to engage a lateral arch of said foot, said lateral arch going along an outside ridge of said foot extending from said ball to a heel of said foot;
a third stimulating mound to engage said lateral arch in front said second stimulating mound behind and outside of said first stimulating mound; and
a fourth stimulating mound to engage said transverse arch near said lateral arch.

39. The shoe of claim 37, wherein said contour further comprises at least two of said members of said stimulating mound group.

40. The shoe of claim 39, wherein said contour further comprises each of said members of said stimulating mound group.

41. The shoe of claim 37, wherein at least one of said stimulating mounds includes an elastic device.

42. The shoe of claim 37, wherein said contour, further comprises: a heel ridge configured to urge said heel to separate from the rest of said foot.

43. The shoe of claim 42, said heel ridge further comprises: at least two heel ridge components.

44. The shoe of claim 37, wherein said shoe is at least one of a boot, a lace-up shoe, a sandal, and a mule.
